(19) United States
(12) Patent Application Publication
(10) Pub. No.: US 2025/0255509 A1
(43) Pub. Date: Aug. 14, 2025

(54) MAGNETIC POSITION SENSOR

(71) Applicant: St. Jude Medical International Holding S.á r.l, Luxembourg (LU)

(72) Inventors: Troy Tegg, Elk River, MN (US); Erich Stoermer, Plymouth, MN (US)

(21) Appl. No.: 19/052,932

(22) Filed: Feb. 13, 2025

Related U.S. Application Data
(60) Provisional application No. 63/553,414, filed on Feb. 14, 2024.

(51) Int. Cl. A61B 5/06; G01D 5/20

(52) U.S. Cl. CPC A61B 5/062; G01D 5/20

(57) ABSTRACT

A magnetic position sensor for tracking a position of a medical device. The magnetic position sensor includes a magnetically permeable core. The magnetically permeable core has a longitudinal axis and a first flat surface extending parallel with the longitudinal axis. A coil including a conductive wire is wrapped around the longitudinal axis of the magnetically permeable core in a plurality of windings.

### CROSS-REFERENCE TO RELATED APPLICATION

This application claims benefit of priority to U.S. Provisional Patent Application No. 63/553,414 filed on Feb. 14, 2024, the entire disclosure of which is hereby incorporated by reference.

### BACKGROUND

The present disclosure relates generally to a magnetic position sensors for tracking a position and/or an orientation of a medical device. In general, medical positioning systems are used to track a position and/or an orientation of a medical device within a patient. Exemplary medical devices used with medical positioning systems include catheters, introducers, guide wires, etc. Such medical devices may include an elongate flexible shaft and various diagnostic and/or therapeutic elements that are used to perform various diagnosis or treatment procedures including mapping and/or ablation on anatomy (e.g., cardiac tissue).

### SUMMARY

According to some embodiments, a magnetic position sensor for tracking a position of a medical device. The magnetic position sensor includes a magnetically permeable core. The magnetically permeable core has a longitudinal axis and a first flat surface extending parallel with the longitudinal axis. A coil including a conductive wire is wrapped around the longitudinal axis of the magnetically permeable core in a plurality of windings.

A method of assembling a magnetic position sensor. The method includes forming a magnetically permeable core from one or more Metglas layers. The magnetically permeable core includes a longitudinal axis and a first flat surface extending parallel with the longitudinal axis. A conductive wire is wound around the longitudinal axis of the magnetically permeable core to form a coil. A pair of wires is electrically coupled to the conductive wire.

A medical device. The medical device includes an elongate shaft including a side wall. The medical device includes a magnetic position sensor. The magnetic position sensor includes a magnetically permeable core including a longitudinal axis and a first flat surface extending parallel with the longitudinal axis. A coil including a conductive wire is wrapped around the longitudinal axis of the magnetically permeable core in a plurality of windings. The magnetic position sensor is located in the side wall of the elongate shaft.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1A is a top view of a flat magnetic position sensor, according to some embodiments.

FIG. 1B is a side view of a flat magnetic position sensor, according to some embodiments.

FIG. 1C is a front view of a flat magnetic position sensor, according to some embodiment.

FIG. 2 is a high-density grid electrode assembly of an example medical catheter that can include one or more flat magnetic position sensor(s), according to some embodiments.

FIG. 3 is an exemplary medical catheter that can include one or more flat magnetic position sensor(s), according to some embodiments.

FIG. 4A is an isometric view of an introducer catheter with a portion of the catheter wall cut away for viewing purposes, according to some embodiments.

FIG. 4B is a cross-sectional side view of an introducer catheter with a flat magnetic sensor located in the catheter wall, according to some embodiments.

FIG. 5 is a medical device localization system that can be employed with a medical device that includes one or more flat magnetic position sensor(s), according to some embodiments.

FIG. 6 is a medical localization system that can be employed with a medical device that includes one or more flat magnetic position sensor(s), according to some embodiments.

FIG. 7 illustrates a flow chart of a method for assembling a flat magnetic position sensor, according to some embodiments.

### DETAILED DESCRIPTION

This disclosure relates to devices, systems, and methods for tracking a position and/or an orientation of a medical device with a flat magnetic sensor. The flat magnetic sensor includes a magnetically permeable core having at least one flat (or substantially flat) surface. The flat magnetic sensor may be optimized for space optimization (i.e., efficient utilization of volume) within the medical device. For example, catheters or other intravascular devices have small cross sectional profiles to navigate through vasculature, and therefore, the components/features within the medical device may be arranged to optimize the limited volume provided by the medical device. In some cases, the flat magnetic sensor may be beneficial when the medical device has limited space in one direction (i.e., the height direction of the flat magnetic sensor) and ample space in an orthogonal direction (i.e., the width direction of the flat magnetic sensor). The flat magnetic sensor can be placed within a distal feature of a diagnostic and/or therapeutic delivery catheter. For example, the flat magnetic sensor can be embedded within a wall (e.g., an interior support wall or a side wall) of an electrophysiology mapping catheter to measure a generated magnetic field to determine position and/or orientation of the flat magnetic sensor. In some embodiments, the flat magnetic sensor is positioned within an introducer or introducer catheter, for instance, within a wall of the introducer catheter (e.g., the Agilis™ NXT Steerable Introducers, commercially available from Abbott Laboratories or as seen generally by reference to U.S. Pat. No. 7,914,515 entitled “Catheter and introducer catheter having torque transfer layer and method of manufacture” to Heideman et al.).

The flat magnetic sensor may be sensitive to magnetic fields orthogonal to the one or more flat surfaces of the flat magnetically permeable core. For example, if a flat surface of the flat magnetically preamble core is orthogonal to an emitted magnetic field, the flat surface can receive the magnetic field through the flat surface and concentrate the magnetic field through the coil. Because the flat surface of the flat magnetically permeable core has a large width to height ratio (as compared to a cylindrical core which is 1:1), the flat magnetically permeable core is more sensitive to the magnetic field per unit of volume (i.e., cross-sectional area of cylindrical core is d²/4 whereas cross sectional area of a thin, flat rectangular core is d×h, wherein d is width and h is height).

The flat magnetically preamble core may be formed of Metglas (a thin amorphous metal alloy ribbon produced by using rapid solidification process of approximately 1,000,000° C./s). Metglas has a maximum relative permeability of approximately 1,000,000 (μ/μ₀), making it the highest known magnetically permeable material (far greater than standard magnetically permeable cores such as nickel-iron soft ferromagnetic alloy, mu-metal having a maximum relative permeability of approximately 50,000 to 100,000).

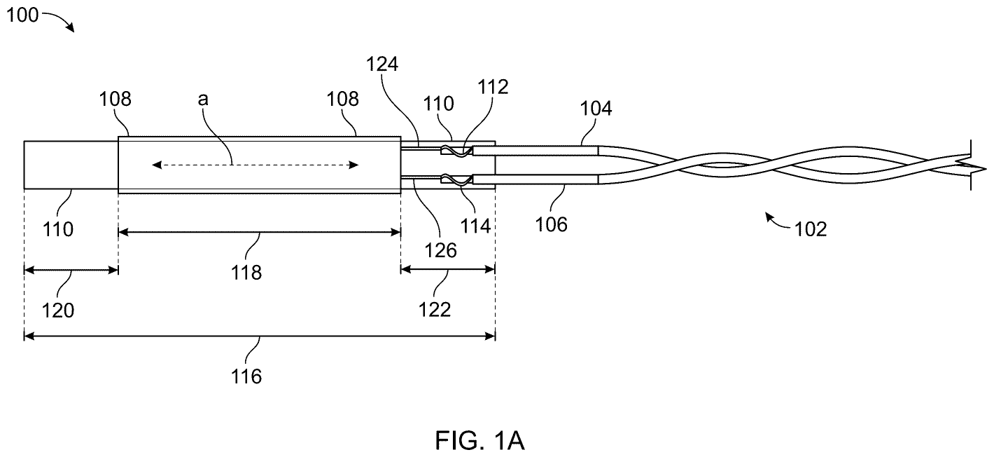

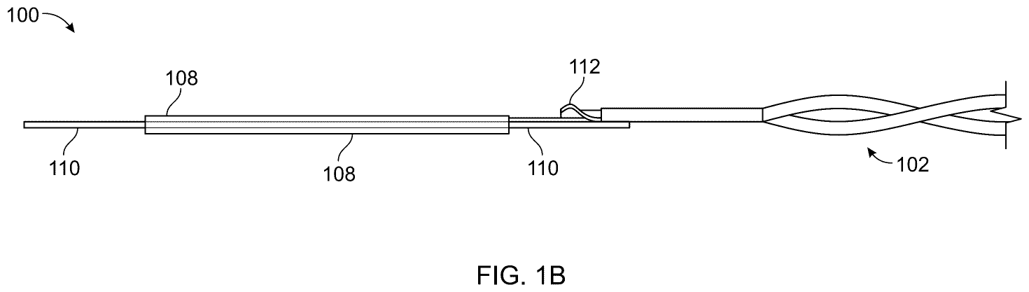

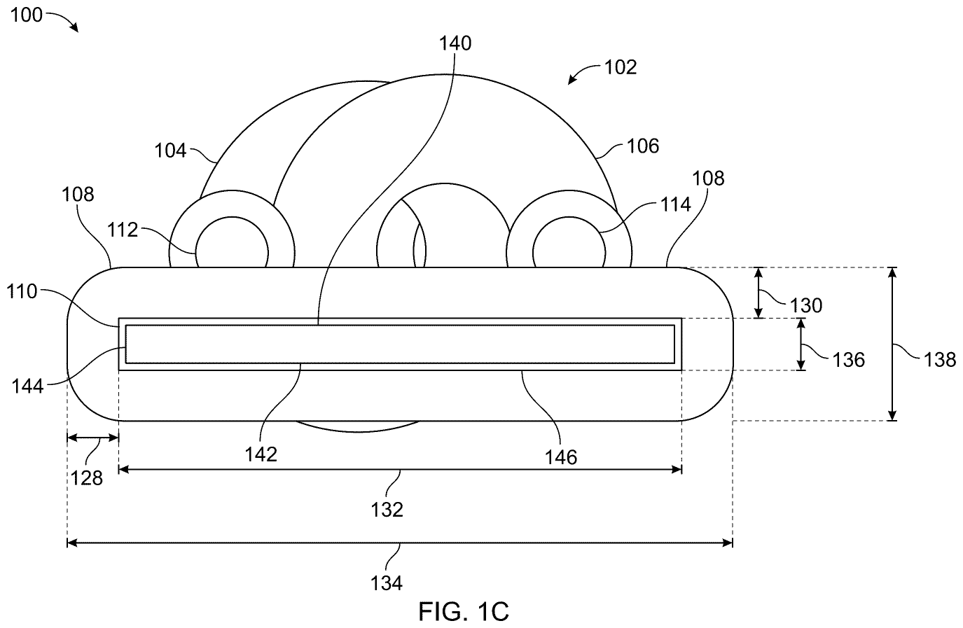

FIG. 1A illustrates a top view of a flat magnetic position sensor 100, according to some embodiments. In some embodiments, the flat magnetic position sensor 100 is connected to a pair of connection wires 102 including a first connection wire 104 and a second connection wire 106. In some embodiments, the flat magnetic position sensor 100 includes a coil 108 wound around a magnetically permeable core 110, a first connection joint 112 and a second connection joint 114, and a first core wire 124 and a second core wire 126. The magnetically permeable core 110 includes a core length 116, a core extension distance 120, and a proximal end distance 122. The coil 108 includes a coil length 118.

The pair of connection wires 102 including the first wire 104 and the second wire 106 may be in a twisted pair configuration. For example, in many embodiments, the pair of connection wires 102 are intertwisted in a range of 10 to 30 turns per inch length, and in some embodiments, the pair of connection wires 102 are intertwisted in a range of 18 to 20 turns per inch length. The pair of connection wires 102 is configured to transmit electrical signals generated by the flat magnetic position sensor 100 in response to a magnetic field generated by a magnetic positioning system (not shown). In some embodiments, the ends of the pair of connection wires 102 are stripped and tinned to improve connection with the first connection joint 112 and the second connection joint 114.

The pair of connection wires 102 including the first wire 104 and the second wire 106 are electrically coupled to the first coil wire 124 and the second coil wire 126 at the first connection joint 112 and the second connection joint 114, respectively. In some embodiments, the first connection joint 112 and the second connection joint 114 (collectively referred to as the connection joints 112, 114) each include solder, and in some embodiments, the solder completely encapsulates each of the first wire 104 and the second wire 106 at the connection joints 112, 114. In some embodiments, the first coil wire 124 and the second coil wire 126 each include a service loop (i.e., excess coil wire in loop). Each of the first coil wire 124 and the second coil wire 126 overlap with the first wire 104 and second wire 106, respectively. In some embodiments, a conductor of the first coil wire 124 contacts a conductor of the first wire 104 and/or solder in-contact with the first wire 104 and a conductor of the second coil wire 126 contacts a conductor of the second wire 106 and/or solder in-contact with the second wire 106. In some embodiments, there is a gap between the connection joints 112, 114 and the coil 108 and/or a gap between the first and second wire 104, 106 and the coil 108. In some embodiments, the connection joints 112, 114 include a weld strain relief to prevent disconnection of the first and second wires 104, 106. The weld strain relief includes an adhesive layer and/or a wire

The coil 108 includes a conductive wire wound or wrapped around a longitudinal axis a of the magnetically permeable core 110. For example, the coil includes a 53 AWG wire with 128 turns (or windings) per layer, with 256 turns total (2 layers, a first layer winding toward the distal end and a second layer winding back toward the proximal end), according to some embodiments. The coil 108 may generate a sensor output voltage of between 1-22 mV, and in some embodiments, may generate a sensor output voltage of between 5-8 mV when tested at 9kH. In some embodiments, the coil 108 may be coated with a layer of UV cure adhesive after the coil 108 is wound around the magnetically permeable core 110.

The magnetically permeable core 110 includes a core length 116 extending along the longitudinal axis a. The coil length 118 is less than the core length 116, and in some embodiments, the magnetically permeable core 110 includes a core extension distance 120 and a proximal end distance 122. In some embodiments, the coil length 118 is less than 83% of the core length 116. For example, in the embodiment illustrated in FIGS. 1A-C, the coil length 118 is approximately 60% of the core length 116 (e.g., the coil length is 0.090″ and the core length is 0.150″). The reduced longitudinal length of the coil 108 may be especially beneficial in a magnetic position sensor with a relatively small outer diameter (e.g., on the order of 1 French (0.33 millimeters) or less) in which small diameter wire (e.g., 58 AWG) is used to form the coil in order to keep the electrical resistance of the coil 108 below a suitable limit. The extension of the magnetically permeable core 110 beyond the coil 108 serves to concentrate magnetic field through the coil 108, which increases the resulting voltage induced in the coil 108. For example, magnetic field lines that interact with the extension (the core extension distance 120) are directed into the coil 108 to increase the voltage response (e.g., intensify the sensitivity) of the coil 108, thereby boosting the signal to noise ratio of the flat magnetic sensor 100.

In some embodiments, the core extension distance 120 to coil length 118 ratio is between 1:2 and 1:10, and in some embodiments, the core extension distance 120 to coil length 118 ratio is between 1:3 and 1:7. For example, in the embodiment shown in FIGS. 1A-C, the core extension distance 120 to coil length 118 ratio is approximately 1:3 (e.g., the core extension distance is 0.030″ and the coil length is 0.090″). In some embodiments, the proximal end distance 122 to coil length 118 ratio is between 1:2 and 1:10, and in some embodiments, the proximal end distance 122 to coil length 118 ratio is between 1:3 and 1:7. For example, in the embodiment shown in FIGS. 1A-C, the proximal end distance 122 to coil length 118 ratio is approximately 1:3 (e.g., the proximal end distance is 0.030″ and the coil length is 0.090″). In some embodiments, the coil 108 is centered on the magnetically permeably core 110 such that the core extension distance 120 is approximately equal to the proximal end distance 122. In some embodiments, the magnetically permeable core 110 does not extend beyond the coil 108 (e.g., the magnetically permeable core 110 extends an equal distance to the coil 108), and in some embodiments, the coil 108 may extend further than the magnetically permeable core 110.

FIG. 1B illustrates a side view of the flat magnetic position sensor 100, according to some embodiments. In some embodiments, the connection joints 112, 114 are secured to a top surface of the magnetically permeable core 110.

FIG. 1C illustrates a front view of the flat magnetic position sensor 100, according to some embodiments. In some embodiments, the magnetically permeable core 110 includes a first flat surface 140 and a second flat surface 142 parallel to the first flat surface 140. In some embodiments, the magnetically permeable core 110 includes one or more third flat surfaces 144 positioned between the first flat surface 140 and the second flat surface 142, for example, forming a rectangular cross sectional profile. Other cross sectional profiles are contemplated, including a substantially flat ellipse, an oval, a rectangle with rounded edges, etc.). In some embodiments, the proximal end of the magnetically permeable core 110 supports the first connection joint 112 and the second connection joint 114. In some embodiments, the magnetically permeable core 110 is coated with a polyethylene terephthalate (PET) layer 146.

The magnetically permeable core 110 includes a height 136 and a width 132. In some embodiments, the magnetically permeable core 110 is a substantially thin and flat shape including a height to width ratio (i.e., the height 136 over the width 132) less than 1:1. In some embodiments the height to width ratio of the magnetically permeable core 110 is between 1:4 and 1:50, in some embodiments the height to width ratio of the magnetically permeable core 110 is between 1:6 and 1:35, and in some embodiments the height to width ratio of the magnetically permeable core 110 is between 1:8 and 1:25. In some embodiments, including the example illustrated in FIG. 1C, the height to width ratio of the magnetically permeable core 110 is preferably between 1:11 and 1:20 (e.g., the height is 0.0010″±0.0002″ and the width is 0.015″±0.001″).

The coil 108 includes a height 138 and a width 134. In some embodiments, the coil 108 includes a coil thickness 128, 130 dependent upon one or more of the thickness of the coil wire, the number of windings, and/or the number of winding layers. For example, some embodiments the coil wire is 53 AWG with 128 turns (or windings) per layer, with 2 total layers (a first layer winding toward the distal end and a second layer winding back toward the proximal end), resulting in the coil thickness 128, 130 approximately equal to 0.0015″. In some embodiments the height to width ratio of the coil 108 is between 1:2 and 1:30, in some embodiments the height to width ratio of the coil 108 is between 1:3 and 1:20, and in some embodiments the height to width ratio of the coil 108 is between 1:4 and 1:12. In some embodiments, including the example illustrated in FIG. 1C, the height to width ratio of the coil 108 is between 1:4 and 1:5 (e.g., the height is 0.004″ and the width is 0.018″=0.001″).

In some embodiments, the magnetically permeable core 110 includes and/or is formed from a Metglas material (a thin amorphous metal alloy ribbon produced by using rapid solidification process of approximately 1,000,000° C./s). Metglas has a maximum relative permeability of approximately 1,000,000 (μ/μ0), making it one of the highest known magnetically permeable material (far greater than standard magnetically permeable cores such as nickel-iron soft ferromagnetic alloy, mu-metal having a maximum relative permeability of approximately 50,000 to 100,000). The rapid solidification process used to create Metglas creates thin ribbons of Metglas which are difficult (or impossible) to form into cylindrical structures. Metglas is typically manufactured as a thin sheet. In some embodiments, one or more layers of Metglas are stacked and pressed to form the flat magnetically permeable core 110. The flat magnetically permeable core 110 including Metglas enhances the signal to noise ratio of the flat magnetic sensor 100 and thereby enhances the reliability and accuracy of the magnetic positioning system.

In some embodiments, the flat magnetic sensor 100 provides a higher sensitivity detection of rotation than a cylindrical magnetic sensor. For example, in a standard cylindrical magnetic sensor, rotation of the sensor (i.e., radial rotation with the longitudinal axis of the cylinder magnetic sensor orthogonal to the magnetic field) may not be detected, as the voltage induced on the coil remains approximately constant. In contrast, the flat magnetic sensor is not a uniform shape about the longitudinal axis and therefore rotation of the sensor is detectable. In some embodiments, the flat magnetic sensor 100 expresses position and orientation data with six degrees-of-freedom (six DOF) as a 3D position (e.g., X, Y, Z coordinates) and 3D orientation (e.g., roll, pitch, and yaw).

In some embodiments, the flat magnetic sensor 100 may be sensitive to magnetic fields orthogonal to the one or more flat surfaces 140, 142 of the flat magnetically permeable core 110. For example, if a flat surface of the flat magnetically permeable core is orthogonal to an emitted magnetic field, the flat surface can receive the magnetic field through the flat surface and concentrate the magnetic field through the coil. Because the flat surface of the flat magnetically permeable core has a large width to height ratio (as compared to a cylindrical core which is 1:1), the flat magnetically permeable core is more sensitive to the magnetic field per unit of volume (i.e., cross-sectional area of cylindrical core is d²/4 whereas cross sectional area of a thin, flat rectangular core is d×h, wherein d is width and h is height).

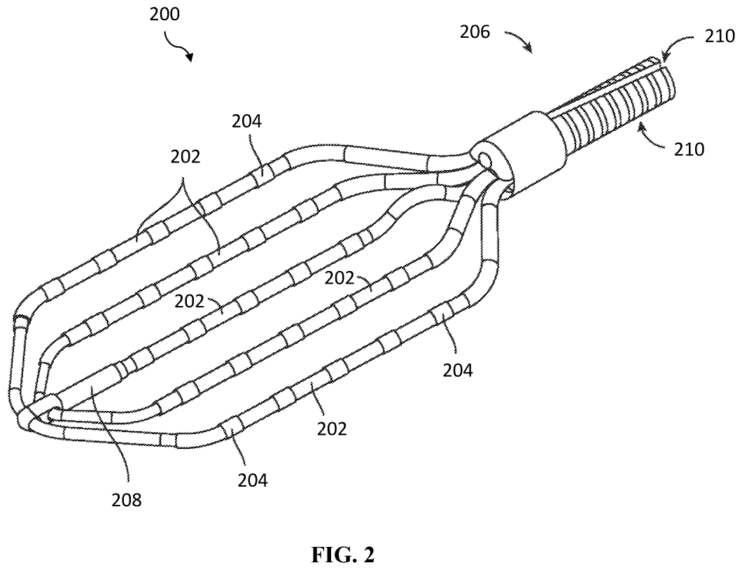

FIG. 2 illustrates high-density grid electrode assembly 200 of an example medical catheter that can include one or more instances of the flat magnetic position sensor 100. The electrode assembly 200 includes five flexible splines 202 and spaced apart electrodes 204. Each of the flexible splines 202 supports five of the electrodes 204. The electrode assembly 200 is configured to self-expand from a collapsed delivery configuration wherein the flexible splines 202 are constrained within a lumen of an introducer catheter to the expanded configuration shown in FIG. 2. The flexible splines 202 have a bending compliance that accommodates conforming the splines 202 to a tissue surface, such as an interior surface of a heart to place each of the electrodes 204 in contact with the tissue surface for using the electrodes 204 to perform a diagnostic and/or therapeutic medical procedure on the tissue. The electrode assembly 200 is mounted to the distal end of an elongate catheter shaft assembly 206. As described herein, the flat magnetic position sensor 100 can have a small cross sectional profile that accommodates installation of the magnetic position sensor 200 within any one or more of the flexible splines 202. For example, an instance of the flat magnetic position sensor 100 can be installed within a distal end portion 208 of the central flexible spline 202. An instance of the flat magnetic position sensor 100 can be installed within a lumen of any of the flexible splines 202 at a suitable longitudinal location, such as, for example, between adjacent instances of the electrodes 204. One or two instances of the flat magnetic position sensor 100 can be installed in slots 210 in the distal end of the elongate catheter shaft assembly 206. The instances of the flat magnetic position sensor 100 included in the electrode assembly 200 can be used to generate signals indicative of the position and/or orientation of the corresponding locations of the electrode assembly 200 within a patient using a medical positioning system as described herein.

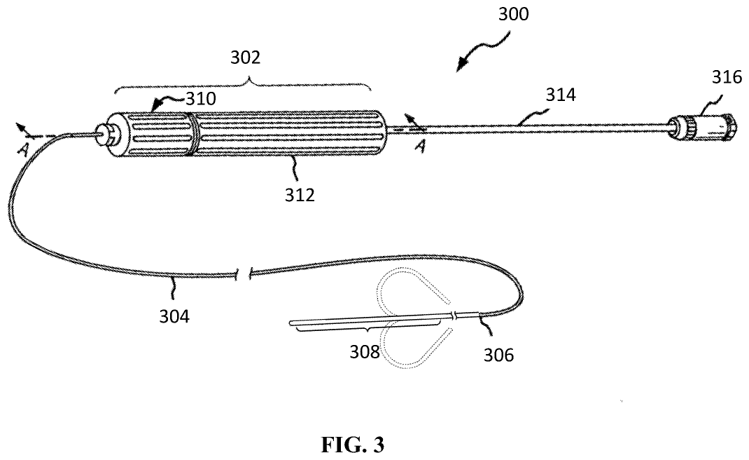

FIG. 3 illustrates an exemplary medical catheter 300 that can include one or more instances of the flat magnetic position sensor 100. The catheter 300 includes a handle assembly 302 and an elongated shaft assembly 304. The shaft assembly 304 includes a flexible shaft 306 and a steerable section 308. The handle assembly 302 is drivingly coupled with the steerable section 308 and operable to selectively bend the steerable section 308 in two directions. As described herein, the flat magnetic position sensor 100 can have a small cross sectional profile that accommodates installation of the flat magnetic position sensor 100 within and/or distal to the steerable section 308. The instances of the flat magnetic position sensor 100 included in the catheter 300 can be used to generate signals indicative of the position and/or orientation of corresponding locations of the catheter 300 within a patient using a medical positioning system as described herein.

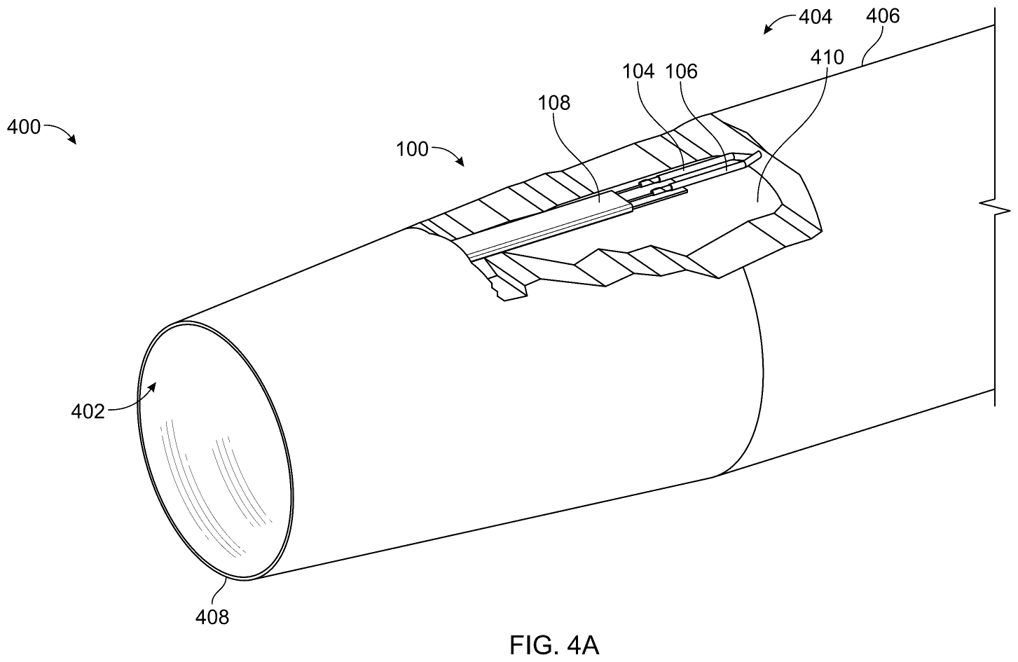

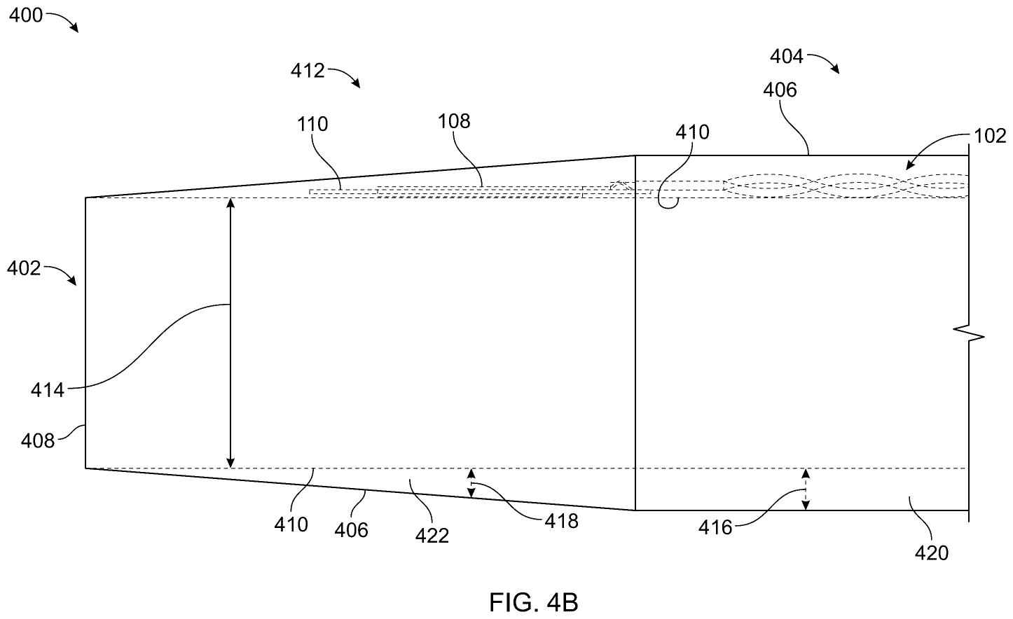

FIG. 4A is an isometric view of an exemplary introducer catheter 400, according to some embodiments. The introducer catheter 400 includes an elongate shaft 404 with a lumen 402 extending therethrough, an outer wall 406 and an inner wall 410 defining a catheter side wall therebetween, and a distal end 408. In some embodiments, the introducer catheter 400 is steerable and configured to deliver a medical instrument through the lumen 102 (e.g., the Agilis™ N×T Steerable Introducers, commercially available from Abbott Laboratories or as seen generally by reference to U.S. Pat. No. 7,914,515 entitled “Catheter and introducer catheter having torque transfer layer and method of manufacture” to Heideman et al.).

FIG. 4B is a cross-sectional view of the introducer catheter 400, according to some embodiments. The introducer catheter 400 includes a tapered distal portion 412 wherein the outer wall 406 converges toward the inner wall 410. In some embodiments, a lumen diameter 414 remains approximately constant within the tapered distal portion. A first wall 420 of the elongate shaft 404 (i.e., the wall between the inner wall 410 and the outer wall 406 on the elongate shaft 404) has a first wall thickness 416. A distal portion wall 422 of the tapered distal portion 412 (i.e., the wall between the inner wall 410 and the outer wall 406 on the tapered distal portion 412) has a second wall thickness 418. The second wall thickness 418 gradually reduces toward the distal end 408.

In some embodiments, the flat magnetic sensor 100 is positioned between the inner wall 410 and the outer wall 406. For example, the flat magnetic sensor 100 is disposed within the first wall 420 of the elongate shaft 404 and/or within the distal portion wall 422 of the tapered distal portion 412. The pair of wires 102 are positioned between the inner wall 410 and the outer wall 406. The substantially flat and thin profile of the flat magnetic sensor 100 provides space optimization (i.e., efficient utilization of volume) within the catheter 400. For example, the wall thicknesses 416, 418 may be much too small for a conventional cylindrical magnetic sensor (i.e., there is limited space in the dimension between the inner wall 410 and the outer wall 406). However, there is ample space in the orthogonal direction (i.e., along the circumference of the catheter 400). Therefore, the flat magnetic sensor 100 is beneficial, as it has a narrow profile to fit within the thin walls 420, 422, and a wide profile along the circumference of the catheter 400 to enhance detection of magnetic fields.

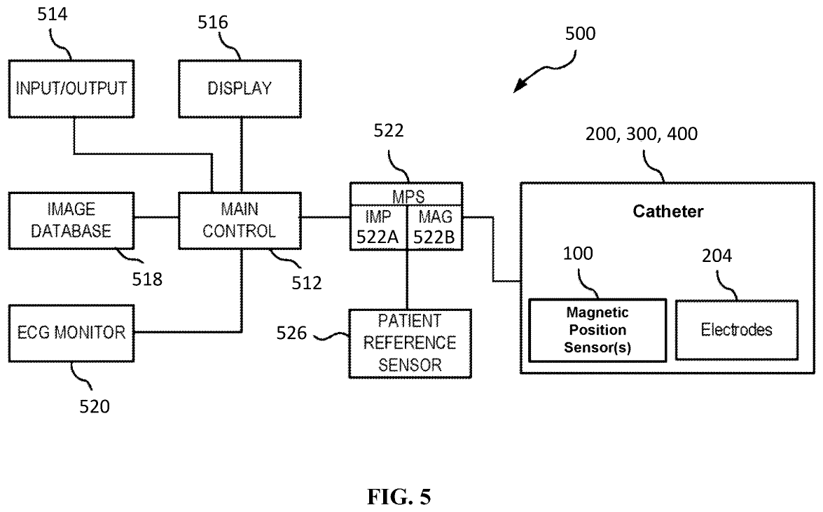

FIG. 5 is a diagrammatic view of a medical device localization system 500 that can be used in conjunction with the flat magnetic position sensor(s) 100. The system 500 includes a main electronic control unit 512 (e.g., a processor) having various input/output mechanisms 514, a display 516, an optional image database 518, an electrocardiogram (ECG) monitor 520, a localization system, such as a medical positioning system 522, and a catheter 200, 300, 400. As described herein, in some embodiments the catheter 200, 300, 400 includes the electrodes 204 and one or more of the flat magnetic position sensors 100.

The input/output mechanisms 514 may include conventional apparatus for interfacing with a computer-based control unit including, for example, one or more of a keyboard, a mouse, a tablet, a foot pedal, a switch and/or the like. The display 516 may also comprise conventional apparatus, such as a computer monitor.

Various embodiments described herein may find use in navigation applications that use real-time and/or pre-acquired images of a region of interest. Therefore, the system 500 may optionally include the image database 518 to store image information relating to the patient's body. Image information may include, for example, a region of interest surrounding a destination site for the catheter 200, 300, 400 and/or multiple regions of interest along a navigation path contemplated to be traversed by the catheter 200, 300, 400. The data in the image database 518 may include known image types including (1) one or more two-dimensional still images acquired at respective, individual times in the past; (2) a plurality of related two-dimensional images obtained in real-time from an image acquisition device (e.g., fluoroscopic images from an x-ray imaging apparatus), wherein the image database 418 acts as a buffer (live fluoroscopy); and/or (3) a sequence of related two-dimensional images defining a cine-loop wherein each image in the sequence has at least an ECG timing parameter associated therewith, adequate to allow playback of the sequence in accordance with acquired real-time ECG signals obtained from the ECG monitor 520. It should be understood that the foregoing embodiments are examples only and not limiting in nature. For example, the image database 518 may also include three-dimensional image data as well. It should be further understood that the images may be acquired through any imaging modality, now known or hereafter developed, for example X-ray, ultra-sound, computerized tomography, nuclear magnetic resonance or the like.

The ECG monitor 520 is configured to continuously detect an electrical timing signal of the heart organ through the use of a plurality of ECG electrodes (not shown), which may be externally affixed to the outside of a patient's body. The timing signal generally corresponds to a particular phase of the cardiac cycle, among other things. Generally, the ECG signal(s) may be used by the control unit 512 for ECG synchronized play-back of a previously captured sequence of images (cine loop) stored in the database 518. The ECG monitor 520 and ECG-electrodes may both include conventional components.

Another medical positioning system sensor, namely, a patient reference sensor (PRS) 526 (if provided in the system 500) can be configured to provide a positional reference of the patient's body so as to allow motion compensation for patient body movements, such as respiration-induced movements. Such motion compensation is described in greater detail in U.S. patent application Ser. No. 12/650,932, entitled “Compensation of Motion in a Moving Organ Using an Internal Position Reference Sensor”, hereby incorporated by reference in its entirety as though fully set forth herein. The PRS 526 may be attached to the patient's manubrium sternum or other location. The PRS 526 can be configured to detect one or more characteristics of the magnetic field in which it is disposed, wherein medical positioning system 522 determines a location reading (e.g., a P&O reading) indicative of the PRS's position and orientation in the magnetic reference coordinate system.

The medical positioning system 522 is configured to serve as the localization system and therefore to determine position (localization) data with respect to the one or more flat magnetic position sensors 100 and/or the electrodes 204 and output a respective location reading. In an embodiment, the medical positioning system 522 may include a first medical positioning system or an electrical impedance-based medical positioning system 522A that determines locations of the electrodes 204 in a first coordinate system, and a second medical positioning system or magnetic field-based medical positioning system 522B that determines location(s) of the flat magnetic position sensor(s) 100 in a second coordinate system. In an embodiment, the location readings may each include at least one or both of a position and an orientation (P&O) relative to a reference coordinate system (e.g., magnetic based coordinate system or impedance based coordinate system). In some embodiments, the P&O may be expressed with five degrees-of-freedom (five DOF) as a three-dimensional (3D) position (e.g., a coordinate in three perpendicular axes X, Y and Z) and two-dimensional (2D) orientation (e.g., a pitch and yaw) of the flat magnetic position sensor(s) 100 in a magnetic field relative to a magnetic field generator(s) or transmitter(s) and/or the electrodes 204 in an applied electrical field relative to an electrical field generator (e.g., a set of electrode patches). In some embodiments, the P&O may be expressed with six degrees-of-freedom (six DOF) as a 3D position (e.g., X, Y, Z coordinates) and 3D orientation (e.g., roll, pitch, and yaw).

The impedance based medical positioning system 522A determines locations of the electrodes 204 based on capturing and processing signals received from the electrodes 204 and external electrode patches while the electrodes 204 are disposed in a controlled electrical field (e.g., potential field) generated by the electrode patches, for example. The MPS system 522A may include various visualization, mapping and navigation components as known in the art, including, for example, an EnSite™ X EP System commercially available from Abbott Laboratories or as seen generally by reference to U.S. Pat. No. 7,263,397 entitled “Method and Apparatus for Catheter Navigation and Location and Mapping in the Heart” to Hauck et al., or U.S. Pat. Publication No. 2007/0060833 A1 to Hauck entitled “Method of Scaling Navigation Signals to Account for Impedance Drift in Tissue”, both owned by the common assignee of the present invention, and both hereby incorporated by reference in their entireties.

The magnetic-based medical positioning system 522B determines locations (e.g., P&O) of the flat magnetic position sensor(s) 100 in a magnetic coordinate system based on capturing and processing signals received from the flat magnetic position sensor(s) 100 while the flat magnetic position sensor 100 is disposed in a controlled low-strength alternating current (AC) magnetic (e.g., magnetic) field. The changing or AC magnetic field may induce a current in the coil(s) 108 when the coil(s) 108 are in the magnetic field. The flat magnetic position sensor(s) 100 is thus configured to detect one or more characteristics (e.g., flux) of the magnetic field(s) in which it is disposed and generate a signal indicative of those characteristics, which is further processed by medical positioning system 522B to obtain a respective P&O for the flat magnetic position sensor(s) 100 relative to, for example, a magnetic field generator.

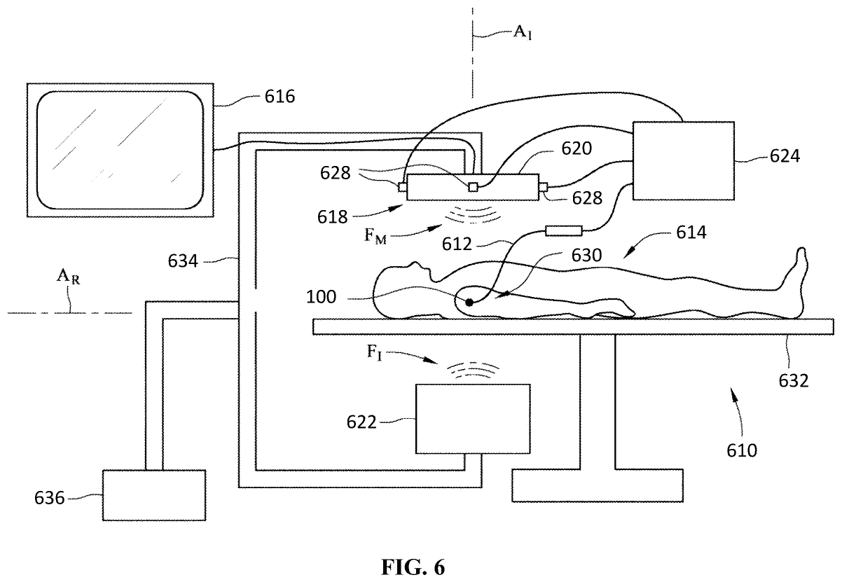

FIG. 6 illustrates another example medical positioning system 610 that can be employed in conjunction with a medical device 612 that includes one or more instances of the flat magnetic position sensor 100 to determine the position and/or orientations of the flat magnetic position sensor(s) 100 within a patient 614 and thereby corresponding location(s) and/or orientations of the medical device 612 within the patient 614. While the medical device 612 is described in the following description as including one magnetic position sensor, the medical device 612 can include more than one instance of the flat magnetic position sensor 100 (e.g., 2, 3, 4, 5, or more instances of the flat magnetic position sensor 100) and the system 610 can be process output from any suitable number of the flat magnetic position sensors 100 to determine the position and/orientation of the flat magnetic position sensors 100. In some embodiments, the system 610 includes a display 616 and is configured to generate and display a model of an internal tissue surface of the patient 614 on the display 616 based on the determined positions and/or orientations of the flat magnetic position sensors 100. The system 610 includes a moving imager 618, which includes an intensifier 620 and an emitter 622, and a magnetic positioning system (MPS) 624, which includes field generators 628. In some embodiments, the combination of the medical device 612 and the system 610 is configured to generate electrophysiology map information and cardiac mechanical activation data pertaining to the tissue model generated by medical imaging system 610 and display the map information and the activation data on the display 616 to facilitate diagnosis and treatment of the patient 614. As described herein, the flat magnetic position sensor 100 may have an improved signal to noise ratio that enhances the accuracy and reliability of the determination of the location and/or orientation of the flat magnetic position sensor 100 by the system 610.

The moving imager 618 acquires an image of a region of interest 630 while the patient 514 lies on an operation table 632. The intensifier 620 and the emitter 622 are mounted on a C-arm 634, which is positioned relative to the patient 614 using a moving mechanism 636. In one embodiment, the moving imager 618 includes a fluoroscopic or X-ray type imaging system that generates a two-dimensional (2D) image of the heart of the patient 614.

The magnetic positioning system (MPS) 624 includes magnetic field generators 628. The MPS 624 determines the position and orientation of the flat magnetic position sensor 100 of the medical device 612 in a coordinate system based on output from the flat magnetic positioning sensor 100 while disposed in magnetic field(s) generated by the magnetic field generators 628.

The C-arm 634 positions the intensifier 620 above the patient 614 and the emitter 622 underneath operation table 632. The emitter 622 generates, and intensifier 620 receives, an imaging field FI, e.g., a radiation field, that generates a 2D image of the area of interest 630 on the display 616. The intensifier 620 and the emitter 622 of the moving imager 618 are connected by the C-arm 634 so as to be disposed at opposites sides of patient 614 along an imaging axis AI, which extends vertically with reference to FIG. 6 in the described embodiment. The moving mechanism 636 rotates the C-arm 634 about a rotation axis AR, which extends horizontally with reference to FIG. 6 in the described embodiment. The moving mechanism 636 or an additional moving mechanism may be used to move the C-arm 634 into other orientations. For example, the C-arm 634 can be rotated about an axis (not shown) extending into the plane of FIG. 6 such that imaging axis A_{I} is rotatable in the plane of FIG. 6. As such, the moving imager 618 can be associated with a three-dimensional imaging coordinate system having an x-axis (Xₚ), a y-axis (Yₚ), and a z-axis (Zₚ).

The magnetic positioning system (MPS) 624 is positioned to allow the medical device 612 and the field generators 628 to interact with the MPS 624 through the use of appropriate wired and/or wireless technology. The medical device 612 is inserted into the vasculature of the patient 614 such that flat magnetic position sensor 100 is located within the area of interest 630. The field generators 628 are mounted to the intensifier 620 so as to be capable of generating a magnetic field (F_{M}) in the area of interest 630 coextensive with the imaging field FI. The MPS 624 is able to detect the position and orientation of the flat magnetic position sensor 100 within the magnetic field (F_{M}).

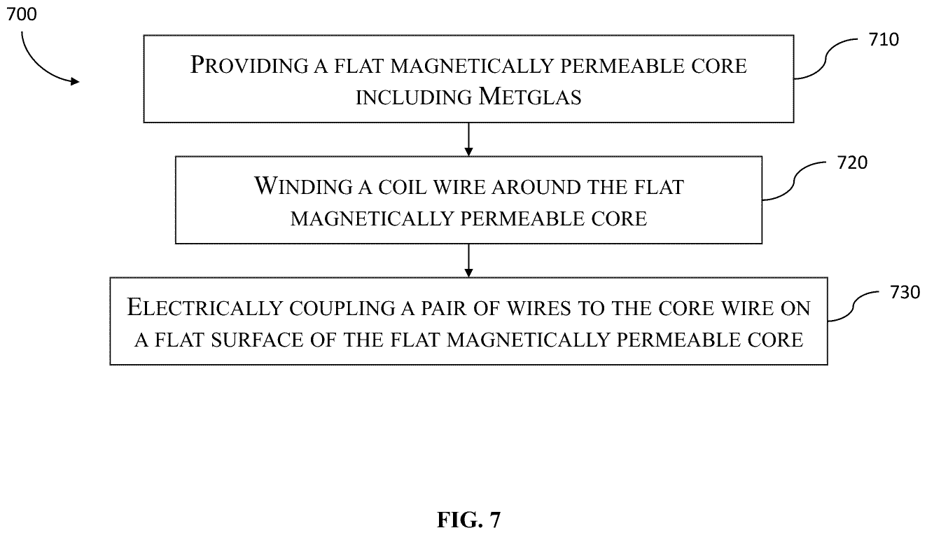

FIG. 7 illustrates a method 700 of assembling a flat magnetic position sensor, according to some embodiments. The method 700 includes step 710, providing a flat magnetically permeable core 110 including Metglas. The method 700 includes step 720, winding a coil wire 124, 126 around the flat magnetically permeable core 110. The method 700 includes step 730, electrically coupling a pair of wires 102 to the core wire 124, 126 on a flat surface 140 of the flat magnetically permeable core 110.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### Discussion of Possible Embodiments

The following are non-exclusive descriptions of possible embodiments of the present invention.

In some aspects, the techniques described herein relate to a magnetic position sensor for tracking a position of a medical device, the magnetic position sensor including: a magnetically permeable core including a longitudinal axis and a first flat surface extending parallel with the longitudinal axis; and a coil including a conductive wire wrapped around the longitudinal axis of the magnetically permeable core in a plurality of windings.

In some aspects, the techniques described herein relate to a magnetic position sensor, further including: a pair of wires electrically coupled to the conductive wire on the first flat surface of the magnetically permeable core.

In some aspects, the techniques described herein relate to a magnetic position sensor, wherein one or more layers of Metglas are stacked to the magnetically permeable core.

In some aspects, the techniques described herein relate to a magnetic position sensor, wherein the one or more layers of Metglas include a plurality of Metglas ribbons.

In some aspects, the techniques described herein relate to a magnetic position sensor, wherein the magnetically permeable core includes a second flat surface extending parallel to the longitudinal axis.

In some aspects, the techniques described herein relate to a magnetic position sensor, wherein the magnetically permeable core includes a core width and a core height, wherein a ratio of core height to core width is within a range of 1:4 and 1:50.

In some aspects, the techniques described herein relate to a magnetic position sensor, wherein the magnetically permeable core extends distal to a distal end of the coil by a core extension distance.

In some aspects, the techniques described herein relate to a magnetic position sensor, wherein a ratio of the core extension distance to a coil length of the coil is within a range of 1:2 and 1:10.

In some aspects, the techniques described herein relate to a magnetic position sensor, wherein the magnetically permeable core extends proximal to a proximal end of the coil.

In some aspects, the techniques described herein relate to a magnetic position sensor, wherein the plurality of windings of the coil extend a coil length along the longitudinal axis and the magnetically permeable core extends a core length along the longitudinal axis, wherein the coil length is less than 83% of the core length.

In some aspects, the techniques described herein relate to a method of assembling a magnetic position sensor, the method including: forming a magnetically permeable core from one or more Metglas layers, the magnetically permeable core including a longitudinal axis and a first flat surface extending parallel with the longitudinal axis; winding a conductive wire around the longitudinal axis of the magnetically permeable core to form a coil; and electrically coupling a pair of wires to the conductive wire.

In some aspects, the techniques described herein relate to a method, wherein the pair of wires are electrically coupled to the conductive wire on the first flat surface of the magnetically permeable core.

In some aspects, the techniques described herein relate to a method, wherein the magnetically permeable core includes a second flat surface extending parallel to the longitudinal axis.

In some aspects, the techniques described herein relate to a method, wherein the first flat surface of the magnetically permeable core defines a first plane and wherein the second flat surface of the magnetically permeable core defines a second plane, wherein the first plane is parallel to the second plane.

In some aspects, the techniques described herein relate to a method, wherein the magnetically permeable core is formed via stacking a plurality of Metglas ribbons.

In some aspects, the techniques described herein relate to a method, wherein the magnetically permeable core includes a core width and a core height, wherein a ratio of core height to core width is within a range of 1:6 and 1:35.

In some aspects, the techniques described herein relate to a method, wherein a distal portion of the magnetically permeable core extends distal to a distal end of the coil by a core extension distance, wherein the distal portion includes a flat distal surface parallel to the longitudinal axis of the magnetically permeable core.

In some aspects, the techniques described herein relate to a medical device, including: an elongate shaft including a side wall; and a magnetic position sensor, including: a magnetically permeable core including a longitudinal axis and a first flat surface extending parallel with the longitudinal axis, and a coil including a conductive wire wrapped around the longitudinal axis of the magnetically permeable core in a plurality of windings, wherein the magnetic position sensor is located in the side wall of the elongate shaft.

In some aspects, the techniques described herein relate to a medical device, wherein the longitudinal axis and the first flat surface are oriented parallel to the elongate shaft.

In some aspects, the techniques described herein relate to a medical device, wherein the first flat surface is oriented to face radially outward relative to the elongate shaft, wherein a pair of wires is electrically coupled to the conductive wire on the first flat surface of the magnetically permeable core.

What is claimed is:

1. A magnetic position sensor for tracking a position of a medical device, the magnetic position sensor comprising:
a magnetically permeable core including a longitudinal axis and a first flat surface extending parallel with the longitudinal axis; and
a coil including a conductive wire wrapped around the longitudinal axis of the magnetically permeable core in a plurality of windings.

2. The magnetic position sensor of claim 1, further comprising:
a pair of wires electrically coupled to the conductive wire on the first flat surface of the magnetically permeable core.

3. The magnetic position sensor of claim 1, wherein one or more layers of Metglas are stacked together to form the magnetically permeable core.

4. The magnetic position sensor of claim 3, wherein the one or more layers of Metglas include a plurality of Metglas ribbons.

5. The magnetic position sensor of claim 1, wherein the magnetically permeable core includes a second flat surface extending parallel to the longitudinal axis.

6. The magnetic position sensor of claim 5, wherein the magnetically permeable core includes a core width and a core height, wherein a ratio of core height to core width is within a range of 1:4 and 1:50.

7. The magnetic position sensor of claim 1, wherein the magnetically permeable core extends distal to a distal end of the coil by a core extension distance.

8. The magnetic position sensor of claim 7, wherein a ratio of the core extension distance to a coil length of the coil is within a range of 1:2 and 1:10.

9. The magnetic position sensor of claim 1, wherein the magnetically permeable core extends proximal to a proximal end of the coil.

10. The magnetic position sensor of claim 1, wherein the plurality of windings of the coil extend a coil length along the longitudinal axis and the magnetically permeable core extends a core length along the longitudinal axis, wherein the coil length is less than 83% of the core length.

11. A method of assembling a magnetic position sensor, the method comprising:
forming a magnetically permeable core from one or more Metglas layers, the magnetically permeable core including a longitudinal axis and a first flat surface extending parallel with the longitudinal axis;
winding a conductive wire around the longitudinal axis of the magnetically permeable core to form a coil; and
electrically coupling a pair of wires to the conductive wire.

12. The method of claim 11, wherein the pair of wires are electrically coupled to the conductive wire on the first flat surface of the magnetically permeable core.

13. The method of claim 11, wherein the magnetically permeable core includes a second flat surface extending parallel to the longitudinal axis.

14. The method of claim 13, wherein the first flat surface of the magnetically permeable core defines a first plane and wherein the second flat surface of the magnetically permeable core defines a second plane, wherein the first plane is parallel to the second plane.

15. The method of claim 11, wherein the magnetically permeable core is formed via stacking a plurality of Metglas ribbons.

16. The method of claim 11, wherein the magnetically permeable core includes a core width and a core height, wherein a ratio of core height to core width is within a range of 1:6 and 1:35.

17. The method of claim 11, wherein a distal portion of the magnetically permeable core extends distal to a distal end of the coil by a core extension distance, wherein the distal portion includes a flat distal surface parallel to the longitudinal axis of the magnetically permeable core.

18. A medical device, comprising:
an elongate shaft including a side wall; and
a magnetic position sensor, including:
a magnetically permeable core including a longitudinal axis and a first flat surface extending parallel with the longitudinal axis, and
a coil including a conductive wire wrapped around the longitudinal axis of the magnetically permeable core in a plurality of windings,
wherein the magnetic position sensor is located in the side wall of the elongate shaft.

19. The medical device of claim 18, wherein the longitudinal axis and the first flat surface are oriented parallel to the elongate shaft.

20. The medical device of claim 18, wherein the first flat surface is oriented to face radially outward relative to the elongate shaft, wherein a pair of wires is electrically coupled to the conductive wire on the first flat surface of the magnetically permeable core.
